(19) United States
(12) Patent Application Publication
(10) Pub. No.: US 2023/0158322 A1
(43) Pub. Date: May 25, 2023

(54) PATIENT TRANSFER ASSEMBLY WITH DETACHABLE SUPPORT SURFACE

(71) Applicant: CEDARS-SINAI MEDICAL CENTER, Los Angeles, CA (US)

(72) Inventor: Wei Gao, Santa Clarita, CA (US)

(73) Assignee: CEDARS-SINAI MEDICAL CENTER, Los Angeles, CA (US)

(21) Appl. No.: 18/011,589

(22) PCT Filed: May 14, 2021
(86) PCT No.: PCT/US2021/032431

§ 371 Date: Dec. 20, 2022

Related U.S. Application Data
(60) Provisional application No. 63/054,590, filed on Jul. 21, 2020.

(51) Int. Cl. A61N 2/02; A61N 2/00

(52) U.S. Cl. CPC A61N 2/02; A61N 2/004

(57) ABSTRACT

A patient transfer assembly can transfer patients (e.g., young, sleeping patients or patients unable to move freely/easily) onto a destination surface (e.g., an imaging table of an imaging device) gently (e.g., with a reduced chance of waking). The assembly can include rollers permitting the assembly to be rolled into a position above or adjacent the destination surface. A support surface upon which the patient rests can be moved to a deployed position (e.g., lowered and/or slid out) and/or the destination surface can be raised until the support surface rests upon the destination surface. The support surface can then be detached, allowing the patient and the support surface to remain on the destination surface without needing to remove the patient from the support surface.

### CROSS REFERENCE TO RELATED APPLICATIONS

The present application claims the benefit of U.S. Provisional Patent Application No. 63/054,590, filed Jul. 21, 2020 and entitled “IMAGING PATIENT TRANSFER CRIB,” which is hereby incorporated by reference in its entirety.

### STATEMENT REGARDING FEDERALLY SPONSORED RESEARCH OR DEVELOPMENT

This invention was made with government support under Grant No. DA050255 awarded by National Institutes of Health. The government has certain rights in the invention.

### TECHNICAL FIELD

The present disclosure relates to medical equipment generally and more specifically to patient transfer devices.

### BACKGROUND

Transporting patients quickly and safely in healthcare facilities can be of great importance. Existing techniques to transport a patient between an origin location and a destination location often result in jostling the patient or other undesirable effects, which can be detrimental to the health of the patient. For example, existing techniques may awaken patients or otherwise disrupt patients, which may delay further diagnostic tests or treatments.

In an example, there are patients who have certain disabilities that prevent them from moving on to a destination surface (e.g., an imaging bed or patient bed) or who would otherwise undergo pain or anguish from the process of moving themselves onto such a surface (e.g., patients with severe burns or trauma).

In an example, certain medical imaging techniques, such as magnetic resonance (MR) imaging and others, use a patient bed or imaging table to support a patient during imaging. In some cases, the imaging table is used to properly position a patient within a sensing area of an imaging device (e.g., within an aperture of an MR machine). Often, imaging equipment can be loud and disruptive. Additionally, it can be important to minimize delay when conducting medical imaging, such as if the imaging is related to a time-sensitive diagnosis or if the imaging device is to be used for subsequent patients.

In an example, when young patients (e.g., infants, toddlers, and the like) are to be imaged, it can be desirable to have the patient be asleep during the imaging procedure. In such cases, it can be useful to allow the patient to fall asleep in a separate room or location prior to being moved to the imaging table. Existing techniques for transferring such a sleeping patient to an imaging table carry a substantial risk of wakening the patient. To ensure the patient is asleep when the imaging is commenced, some approaches may require a patient to be permitted to fall asleep on the imaging table, which can be difficult due to the noise of the equipment and can monopolize use of the imaging equipment for long periods of time.

Whether young or old, it can be useful to be able to transport patients from any given origin location to any given destination location with minimum disruption to the patient.

### SUMMARY

The term embodiment and like terms are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, supplemented by this summary. This summary is a high-level overview of various aspects of the disclosure and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this disclosure, any or all drawings and each claim.

Embodiments of the present disclosure include a patient transfer assembly comprising a support frame. The patient transfer assembly further comprises a set of rollers defining a base. The set of rollers is coupled to the support frame to facilitate movement of the support frame. The patient transfer assembly further comprises a support surface removably coupled to the support frame to support a patient. The support surface is movable with respect to the base between a transport position and a deployed position. The support surface is detachable from the support frame when in the deployed position.

Embodiments of the present disclosure include a method comprising providing a patient transfer assembly for use with a destination surface. The patient transfer assembly comprises a support frame. The patient transfer assembly further comprises a set of rollers defining a base. The set of rollers is coupled to the support frame to facilitate movement of the support frame. The patient transfer assembly further comprises a support surface removably coupled to the support frame to support a patient. The support surface is movable with respect to the base between a transport position and a deployed position. The support surface is detachable from the support frame when in the deployed position. The patient transfer assembly further comprises a receiving space for receiving the destination surface. The receiving space is located below or adjacent to the support surface when the support surface is in the transport position. The support surface rests on the destination surface when in the deployed position. The method further comprises maneuvering the patient transfer assembly towards the destination surface to position the destination surface within the receiving space of the patient transfer assembly. The method further comprises moving the support surface from the transport position to the deployed position such that the support surface rests upon the destination surface. The method further comprises detaching the support surface from the support frame. The method further comprises maneuvering the support frame away from the destination surface.

### BRIEF DESCRIPTION OF THE DRAWINGS

The specification makes reference to the following appended figures, in which use of like reference numerals in different figures is intended to illustrate like or analogous components.

FIG. 1 is a side schematic diagram depicting a patient transfer assembly separate from an imaging machine, according to certain aspects of the present disclosure.

FIG. 2 is a side schematic diagram depicting a patient transfer assembly positioned over an imaging table of an imaging machine, according to certain aspects of the present disclosure.

FIG. 3 is a side schematic diagram depicting a patient transfer assembly with a lowered support surface at an imaging machine, according to certain aspects of the present disclosure.

FIG. 4 is a side schematic diagram depicting a patient transfer assembly separate from an imaging machine after leaving the patient on the imaging table of an imaging machine, according to certain aspects of the present disclosure.

FIG. 5 is a side schematic diagram depicting a patient being imaged by an imaging table, according to certain aspects of the present disclosure.

FIG. 6 is a top schematic view of a patient transfer assembly, according to certain aspects of the present disclosure.

FIG. 7 is a side schematic view of a patient transfer assembly, according to certain aspects of the present disclosure.

FIG. 8 is a side schematic view of a patient transfer assembly with the support surface lowered, according to certain aspects of the present disclosure.

FIG. 9 is a side schematic view of a patient transfer assembly with the support surface detached, according to certain aspects of the present disclosure.

FIG. 10 is a front schematic view of a patient transfer assembly, according to certain aspects of the present disclosure.

FIG. 11 is a side schematic view of a portion of a support surface with an inflated stiffening support, according to certain aspects of the present disclosure.

FIG. 12 is a side schematic view of a portion of a support surface with a deflated stiffening support, according to certain aspects of the present disclosure.

FIG. 13 is a side schematic diagram depicting a patient transfer assembly positioned adjacent a destination surface, according to certain aspects of the present disclosure.

FIG. 14 is a side schematic diagram depicting a patient transfer assembly with its support surface extended over the destination surface, according to certain aspects of the present disclosure.

FIG. 15 is a side schematic diagram depicting a patient transfer assembly spaced apart from a destination surface with its support surface separated and remaining on the destination surface, according to certain aspects of the present disclosure.

FIG. 16 is an overhead schematic diagram depicting a patient transfer assembly with its support surface in a transport position, according to certain aspects of the present disclosure.

FIG. 17 is an overhead schematic diagram depicting a patient transfer assembly with its support surface in a deployed position, according to certain aspects of the present disclosure.

FIG. 18 is an overhead schematic diagram depicting a patient transfer assembly with its support surface separated from the frame, according to certain aspects of the present disclosure.

FIG. 19 is a flowchart depicting a process for using a patient transfer assembly, according to certain aspects of the present disclosure.

### DETAILED DESCRIPTION

Certain aspects and features of the present disclosure relate to a patient transfer assembly, such as a transfer crib. The patient transfer assembly can transfer patients (e.g., young patients, sleeping patients, and/or patients unable to move easily/freely) onto a destination surface (e.g., an imaging table of an imaging device, such as an MR machine) gently (e.g., with a reduced chance of waking a sleeping patient). The patient transfer assembly can include rollers permitting the patient transfer assembly to be rolled into a position above or adjacent the destination surface. Thereafter, a support surface of the patient transfer assembly (e.g., a floor of a transfer crib) can be lowered and/or slid horizontally, and/or the destination surface can be raised, until the support surface rests upon the destination surface. The support surface can then be detached, allowing the patient and the support surface to remain on the destination surface (e.g., remain and be secured to an imaging table) without needing to remove the patient from the support surface.

The patient transfer assembly can be used with any suitable destination surface, such as a table (e.g., an imaging table, an operating table, or a changing table), a bed (e.g., a patient bed), a crib, or the like. In some cases, the destination surface is a flat surface, such as a table or bed. In some cases, the destination surface may be flat and parallel to the floor.

As described herein, the patient transfer assembly can be especially useful for transferring patients onto an imaging table of a medical imager. Examples of suitable imagers include an MR machine, an X-ray machine, a fluoroscope, an X-ray computed tomography (CT) machine, a nuclear imaging machine (e.g., positron emission tomography (PET) machine), and the like. Certain aspects and features of the present disclosure can be especially useful for large imaging machines, thus permitting the patient to be easily moved to the imaging table of the machine, rather than the machine being moved to the patient. Additionally, certain aspects and features of the present disclosure are especially useful for large imaging machines with attached imaging tables.

The patient transfer assembly can include a support frame. In some cases, the support frame further supports a set of walls (e.g., one or more walls), although that need not always be the case. A support surface can be removably coupled to the support frame, such that the support surface is able to move (e.g., vertically and/or horizontally) between a transport position and a deployed position. The support frame can be coupled to a set of rollers (e.g., one or more rollers, such as three or more rollers or four or more rollers), such as via one or more legs or other coupling mechanisms. The set of rollers can define a base of the patient transfer assembly. The patient transfer assembly can maintain stability while its center of mass is maintained over the base. In some cases, such as when a support surface is displaced horizontally when moving between the transport position and the deployed position, one or more static or moving counterweights can be used to ensure the center of mass of the patient transfer assembly remains over the base.

When supporting a set of walls, the support frame can support any number of walls (e.g., fabric walls), but commonly supports four walls. The walls can be made of a breathable material. The walls can take the form of traditional crib walls used on infant cribs, although that need not always be the case. A support surface can be removably coupled to the frame to support a patient thereon. The entire patient transfer assembly can be made of one or more MR-safe materials, such as non-ferromagnetic materials. In some cases, the patient transfer assembly can be made of non-ferromagnetic plastics and fabrics. The support surface can be made of one or more materials that are MR-safe. In some cases, the support surface is made of one or more materials that are transparent or substantially translucent (e.g., radiolucent) to the imager being used, such as an MR machine.

The support surface can be removably coupled to the support frame in any suitable fashion, such as through the use of an intermediary support. For example, the support surface can be removably fastened to an intermediary support, which is then coupled to the support frame. The support surface can be removably coupled to the support frame through the use of any number of suitable fasteners. Examples of suitable fasteners include hook and loop fasteners, zippers (e.g., plastic zippers), hooks (e.g., hooks and eyeholes), and the like. The fasteners used to removably couple the support surface can be MR-safe, and may be transparent or substantially translucent to the imager being used.

The patient transfer assembly can include a receiving space below or adjacent to the support surface for receiving a destination surface (e.g., an imaging table of an imaging device). The receiving space can be an open region below the support surface or adjacent to the support surface when the support surface is in the deployed position. In some cases, the receiving space can be optionally be enclosed by the support frame and/or the walls of the patient transfer assembly. In an example, the patient transfer assembly includes openings to the receiving space in a front wall and/or a rear wall to permit the support frame to be moved into position around the destination surface (e.g., imaging table). In another example, the receiving space is an open region below the support surface, allowing the support frame to be moved into position above the destination surface. In another example, the receiving space is an open region adjacent to (e.g., horizontally offset from and slightly below) the support surface, allowing the support surface to be displaced horizontally to position the support surface over the destination surface.

The support frame can be supported on legs with rollers (e.g., casters). Any number of legs can be used, but in some cases the support frame includes two legs or four legs. In some cases, the receiving space is further defined, at least in part, by the legs supporting the support frame. For example, the support frame can have a width that is greater than a width of the destination surface (e.g., an imaging table) and/or the legs can be spaced apart by a width that is greater than a width of the destination surface (e.g., the imaging table), permitting the patient transfer assembly to be positioned around the sides of the destination surface (e.g., the imaging table).

The patient can be allowed to lie upon, and optionally fall asleep upon, the support surface of the patient transfer assembly while the patient transfer assembly is in a first room. Thereafter, the patient transfer assembly can be rolled into a second room with the destination surface (e.g., a room with the imaging equipment). For example, the patient transfer assembly can be wheeled into position over, and optionally around, an imaging table. The support surface can be moved into the deployed position (e.g., lowered and/or displaced horizontally) and/or the imaging table can be raised until the support surface rests on the imaging table, then the support surface can be detached from the remainder of the patient transfer assembly, allowing the remainder of the patient transfer assembly to be rolled away. The patient and the support surface can then remain on the imaging table, optionally being secured to the imaging table as necessary for the imaging protocol. In some cases, the support surface can be made of material(s) that are flexible, such as flexible enough to permit the support surface to fit within supplemental imaging equipment (e.g., a removable piece of imaging equipment), such as an MR imaging coil (e.g., a head coil or a body coil). In such an example, edges of the support surface that extend beyond the patient may be partially wrapped around the inside of the MR imaging coil for the imaging procedure.

In some cases, the support surface can be made of flexible material(s) and can include one or more stiffening elements. The one or more stiffening elements can increase the resistance of the support surface from flexing. In some example, a stiffening element can be removable and can include a stiff board, a set of stiffening rods or slats, or the like. Such a removable stiffening element can be removed through an opening in a side of the support surface, or can be removably slid into a pocket attached to an underside of the support surface. In some cases, however, the support surface can include internal stiffening elements.

Internal stiffening elements can include any stiffening element that can move between a rigid configuration and a flexible configuration. An example of such a stiffening element is a set of one or more inflatable bladders. The set of one or more inflatable bladders can be arranged within the support surface to resist flexion of the support surface along a length of the support surface, along a width of the support surface, or any combination thereof. The set of inflatable bladders can be arranged such that the support surface is flexible when the one or more inflatable bladders are deflated, but rigid when the one or more inflatable bladders are inflated. In some cases, the set of inflatable bladders can be shaped to urge the patient towards a center of the support surface. If multiple inflatable bladders are used, each inflatable bladder can be fluidly coupled to one another, although that need not always be the case. Inflatable bladders can be inflated through an external port and deflated through the same external port or a separate external port. In an example, a supply of fluid (e.g., air from a separate air pump) can be used to inflate the set of inflatable bladders, while a mechanical release valve can be used to release the fluid (e.g., air), thus permitting the inflatable bladders to deflate. The inflatable bladders can be made of an elastomeric material to facilitate deflation.

In an example, before the patient is placed in the patient transfer assembly, the set of inflatable bladders (e.g., one or more inflatable bladders) of the support surface can be inflated to provide comfort and/or structural support to the support surface. After the support surface has moved to the deployed position to rest upon the destination surface, the one or more inflatable bladders can be deflated, permitting the support surface to more easily be maneuvered and bent. In such cases, deflation of the inflatable bladders can occur before or after the support surface is separated from the remainder of the patient transfer assembly. In some cases, this improved maneuverability of the support surface when deflated can permit the support surface to be fit into imaging equipment (e.g., a removable piece of imaging equipment), such as an MR imaging coil (e.g., a head coil or a body coil).

In some cases, a patient transfer assembly as disclosed herein can be a transfer crib. In some cases, such a transfer crib can be sized for a young patient, such as a patient at or below 3, 2.5, 2, 1.5, 1, or 0.5 years of age. However, in some cases, larger variants of the patient transfer assembly can be used to transfer older patients, such as preadolescents, adolescents, young adults, middle adults, and/or older adults. In these cases, the patient transfer assembly may be used without necessarily having the patient fall asleep in the patient transfer assembly Certain aspects and features of the patient transfer assembly can facilitate moving a patient onto a destination surface (e.g., an imaging table) in a quick and gentle fashion, which can be especially beneficial to patients who may be unable to move themselves onto an imaging bed easily or at all, as well as patients who would otherwise undergo pain or anguish from the process of moving themselves onto an imaging bed (e.g., patients with severe burns or trauma). Such a patient can be transported using a patient transfer assembly as disclosed herein. In some cases, legs of a patient transfer assembly can be extendable, such as through the use of linear actuators, to raise and lower the frame of the crib itself, such as to make it easier to initially have a patient lay on the support surface. The use of a patient transfer assembly as disclosed herein can help reduce the amount of time spent in the room with the imaging machine, such as if the patient would otherwise require a longer period of time to move into position on the imaging bed. Thus, another patient can be using the imaging machine when the patient is moving into position on the patient transfer assembly.

In some cases, an additional support surface can be coupled to the patient transfer assembly after a first support surface is removed. For example, after using a patient transfer assembly to place Patient A on a first destination surface, leaving the first support surface sandwiched between Patient A and the first destination surface, the patient transfer assembly can be moved to a new location and a second support surface can be coupled to the patient transfer assembly to use in the movement of Patient B to a second destination surface. Thus, use of an additional support surface can occur even while a first support surface is unavailable.

These illustrative examples are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative embodiments but, like the illustrative embodiments, should not be used to limit the present disclosure. The elements included in the illustrations herein may not be drawn to scale.

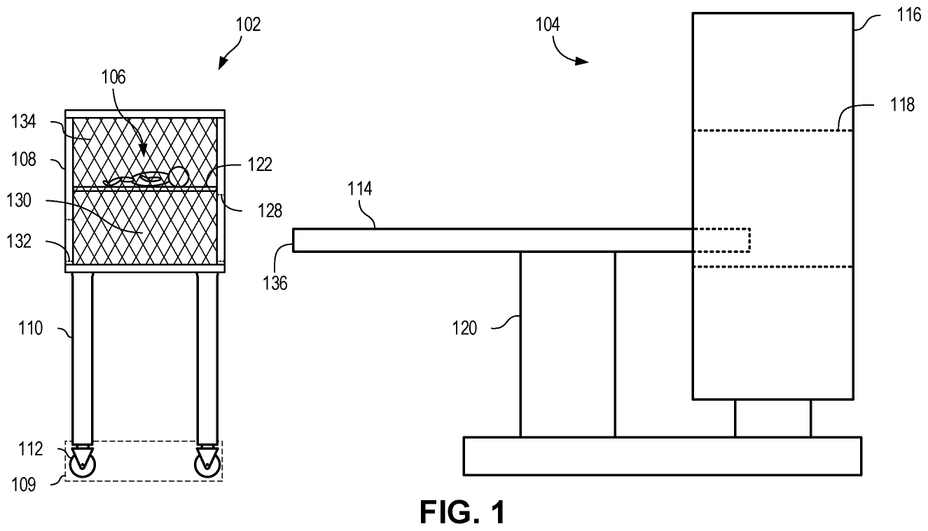

FIG. 1 is a side schematic diagram depicting a patient transfer assembly 102 separate from an imaging machine 104, according to certain aspects of the present disclosure. In some cases, patient transfer assembly 102 can be in a separate environment (e.g., a separate room) from the imaging machine 104, although that need not always be the case. The patient transfer assembly 102 of FIG. 1 is depicted in a crib-like form and used with an imaging machine 104, however that need not always be the case. In some cases, the patient transfer assembly 102 can take another form (e.g., such as a larger, bed-like assembly) and/or can be used with a destination surface that is not part of an imaging machine 104. Thus, in some cases, reference to an imaging machine 104 or imaging table 114 can be replaced with an alternate destination surface.

Imaging machine 104 is depicted as an MR machine, although other machines could be used. The imaging machine 104 can include a main body 116 and an imaging table 114 (e.g., the destination surface). The main body 116 can include an aperture 118 into which the patient can be placed during the imaging procedure. To position and maneuver the patient properly during the imaging procedure, the patient can be placed on and/or secured to an imaging table 114. The imaging table 114 can be supported by an imaging table base 120. In some cases, the imaging table 114 is coupled to the main body 116 of the imaging machine 104, such as by the imaging table base 120 be coupled to a base for the main body 116. The imaging table 114 is movable in a direction parallel to the central axis of the aperture 118. In some cases, the imaging table 114 is adjustable in a vertical direction (e.g., up and down as depicted in FIG. 1).

The patient transfer assembly 102 is designed to be able to be positioned in a room separate from the imaging machine 104, such as to permit the patient 106 to fall asleep, prior to moving the patient transfer assembly 102 into position over the imaging table 114. The patient transfer assembly 102 can include a support frame 108 supporting walls 134 to define the horizontal boundaries of a sleeping area of the patient transfer assembly 102. The walls 134 can be made of any suitable material, such as a fabric and/or polymer material, such as a polymer webbing suitable for baby cribs. The patient transfer assembly 102 may have an open top, although that need not always be the case.

The patient transfer assembly 102 includes a support surface 122, or floor, that supports the patient 106 as the patient 106 is permitted to fall asleep. The support surface 122 is removably coupled to the support frame 108, such as disclosed in further detail herein. The patient transfer assembly 102 can include a receiving space 130 below the support surface 122. The receiving space 130 can receive a portion of the imaging table 114 when the patient transfer assembly 102 is moved into an unloading position to unload the patient 106 onto the imaging table 114. As depicted in FIG. 1, the receiving space 130 is located within the boundaries of support frame 108, although that need not always be the case. In some cases, the bottom edges of support frame 108 can be positioned closer to the support surface 122 such that the receiving space 130 is an open space beneath the support frame 108. When the receiving space 130 is located within the boundaries of support frame 108, the patient transfer assembly 102 can include a front opening 128 and/or a rear opening 132 through which an end 136 of the imaging table 114 can be maneuvered.

The patient transfer assembly 102 can include legs 110 supporting the support frame 108. Any number of legs 110 can be used, such as four legs 110 adjacent four corners of the support frame 108. Rollers 112 (e.g., wheels or casters) can be coupled to the support frame 108 (e.g., via the legs 110) to permit the patient transfer assembly 102 to be easily rolled around on the floor. The rollers 112 can be locking rollers. The rollers 112 can define a base 109 of the patient transfer assembly 102, over which a center of mass of the patient transfer assembly 102 can be maintained.

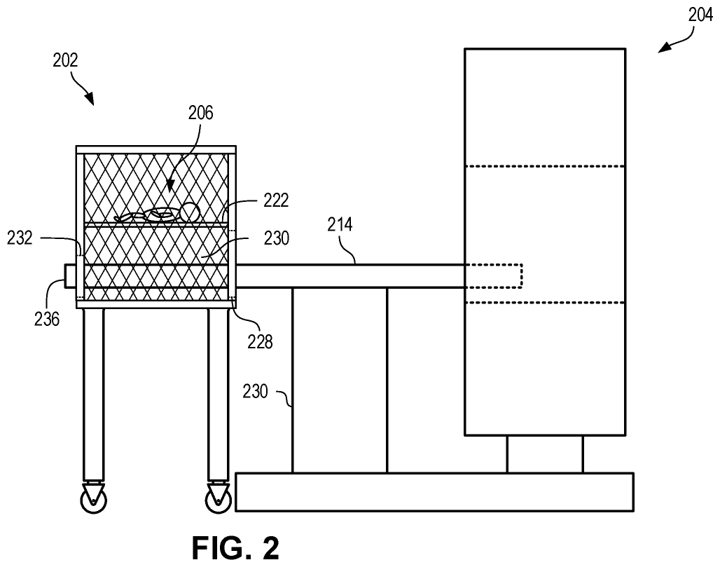

FIG. 2 is a side schematic diagram depicting a patient transfer assembly 202 positioned over an imaging table 214 of an imaging machine 204, according to certain aspects of the present disclosure. Patient transfer assembly 202 and imaging machine 204 can be any suitable patient transfer assembly and destination surface, such as patient transfer assembly 102 and imaging machine 104 of FIG. 1, respectively, after moving the patient transfer assembly into an unloading position. In some cases, reference to an imaging machine 204 or imaging table 214 can be replaced with an alternate destination surface.

In the unloading position, the patient transfer assembly 202 has been moved such that the imaging table 214 of the imaging machine 204 is at least partially within the receiving space 230. As depicted in FIG. 2, an end 236 of the imaging table 214 has been passed through a front opening 228, and optionally a rear opening 232, of the patient transfer assembly 202. In the unloading position, the support surface 222 is positioned above the imaging table 214, thus positioning the patient 206 above the imaging table 214.

In some cases, patient transfer assembly 202 can optionally lock to or engage a destination surface (e.g., via the imaging table base 220), such as to secure the patient transfer assembly 202 in the unloaded position during unloading of the patient 206. In some cases, the support surface 222 is prevented from being lowered and/or detached when the patient transfer assembly 202 is not locked to or engaging the destination surface (e.g., via the imaging table base 220).

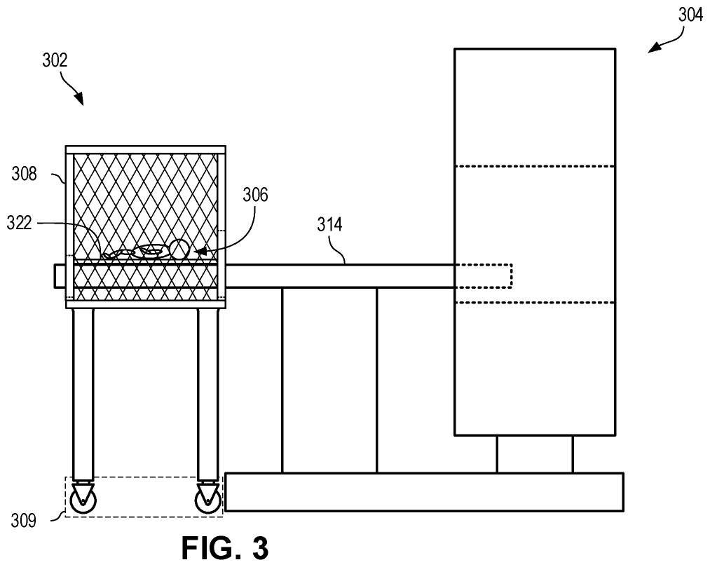

FIG. 3 is a side schematic diagram depicting a patient transfer assembly 302 with a lowered support surface 322 at an imaging machine 304, according to certain aspects of the present disclosure. Patient transfer assembly 302 and imaging machine 304 can be any suitable patient transfer assembly or destination surface, such as patient transfer assembly 202 and imaging machine 204 of FIG. 2, respectively, after lowering the support surface. In some cases, reference to an imaging machine 304 or imaging table 314 can be replaced with an alternate destination surface.

As depicted in FIG. 3, the support surface 322 has been moved from a transport position (e.g., raised, as depicted in FIG. 2) to a deployed position (e.g., lowered, such that it rests upon the imaging table 314). While movement of the support surface 322 of the patient transfer assembly 302 of FIG. 3 involves lowering the support surface 322 in a vertical direction, in some cases, a support surface can be displaced horizontally or lowered and displaced horizontally to move from a transport position to a deployed position.

The support surface 322 can be lowered through any suitable technique, such as through the use of extendable straps, linear actuators, pulleys, and the like. In some cases, the support surface 322 is secured in a raised position (e.g., transport position) until unloading is desired, in which case a user may unsecure the support surface 322 and lower the support surface. For example, support surface 322 can be coupled to straps or other material, such that when the straps or other material is pulled into a first position and secured in place, the support surface 322 is secured in a raised position, but when the straps or other material is unsecured, a user can slowly release tension to allow the support surface 322 to move to a lowered position.

When lowered, the support surface 322 can permit the weight of the patient 306 to be fully supported by the imaging table 314. After the support surface 322 has been lowered, the support surface 322 can be detached, as described in further detail herein, thus permitting the remainder of the patient transfer assembly 402 to be moved away without bothering the patient 306.

Movement of a support surface 322 between a transport position and a deployed position can involve movement of the support surface 322 relative to the base 309. As depicted in FIG. 3, movement of the support surface 322 to the deployed position involves decreasing the distance between the support surface 322 and the base 309 by lowering the support surface 322.

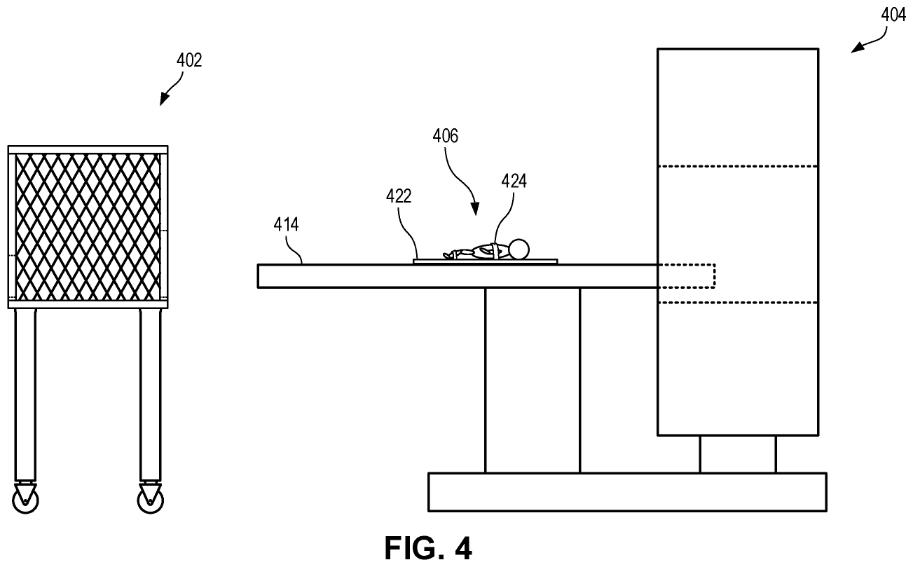

FIG. 4 is a side schematic diagram depicting a patient transfer assembly 402 separate from an imaging machine 404 after leaving the patient on the imaging table of an imaging machine, according to certain aspects of the present disclosure. Patient transfer assembly 402 and imaging machine 404 can be any suitable patient transfer assembly and destination surface, such as patient transfer assembly 302 and imaging machine 304 of FIG. 3, respectively, after detaching the support surface and beginning to move the patient transfer assembly away from the imaging machine. In some cases, reference to an imaging machine 404 or imaging table 414 can be replaced with an alternate destination surface.

After the support surface 422 has been detached from the patient transfer assembly 402, the support surface 422 can remain on the imaging table 414. The patient 406 can be secured to the imaging table 414, such as with straps 424, if suitable for the imaging procedure. The support surface 422 can be made of materials that are safe for use with respect to the imaging machine 404. In some cases, the support surface 422 can be made of materials that are radiolucent with respect to the imaging machine 404, thus permitting the support surface 422 to remain on the imaging table 414, under the patient 406, during the imaging procedures. In some cases, the support surface 422 can be secured to the imaging table 414, along with the patient 406.

Meanwhile, the patient transfer assembly 402 can be moved away from the imaging machine 404, such as to a distant part of the room, or to another room entirely. For example, the patient transfer assembly 402 can be rolled into a control room (e.g., an MR Zone III room) or a changing/holding room (e.g., an MR Zone II room) while the patient 406 is undergoing imaging procedures in the room with the imaging machine 404 (e.g., an MR Zone IV room).

In some cases, after the imaging procedures have been completed, the patient transfer assembly 402 can be moved back to the imaging table 414 to pick up the patient 406, such as be reattaching and raising the support surface 422. However, because there may be no need to gently move the patient 406 after the conclusion of the imaging procedures, and in fact it may be desirable to wake up the patient 406 at such times and have a caregiver hold the patient 406, the patient transfer assembly 402 may not be needed to remove the patient 406 from the imaging device 404.

In some cases, an additional support surface can be coupled to the patient transfer assembly 402, permitting the patient transfer assembly 402 to be used for another patient. Use of such an additional support surface can even occur while the original support surface 422 is still underneath the original patient 406 or otherwise unavailable (e.g., being cleaned). Thus, a single patient transfer assembly 402 can be used to transfer multiple patients to one or more imaging machines 404 without the need to wait until completion of the imaging procedures. In some cases, the use of additional support surfaces can also permit support surfaces to be cleaned and/or sterilized without bringing the patient transfer assembly 402 out of duty.

While not depicted in FIG. 4, additional imaging equipment (e.g., removable imaging equipment) can be coupled to the imaging table to facilitate the imaging procedure, such as receiver coils of an MR machine.

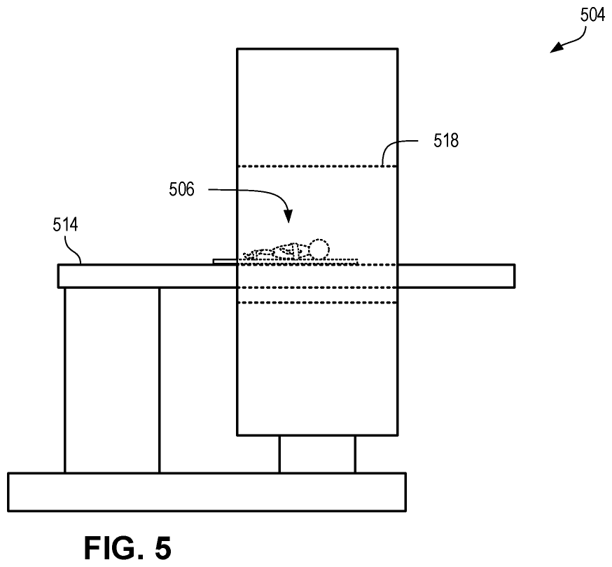

FIG. 5 is a side schematic diagram depicting a patient 506 being imaged by an imaging machine 504, according to certain aspects of the present disclosure. Imaging machine 504 can be imaging machine 404 from FIG. 4. In some cases, reference to an imaging machine 504 or imaging table 514 can be replaced with an alternate destination surface.

During the imaging procedure, the patient 506 can be moved into the aperture 518 of the imaging machine 504 by imaging table 514. Because of the patient transfer assembly's ability to gently place the patient 506 on the imaging table 514, the patient 506 can remain asleep when the imaging procedures commence. While not depicted in FIG. 5, additional imaging equipment (e.g., removable imaging equipment) can be coupled to the imaging table to facilitate the imaging procedure, such as receiver coils of an MR machine.

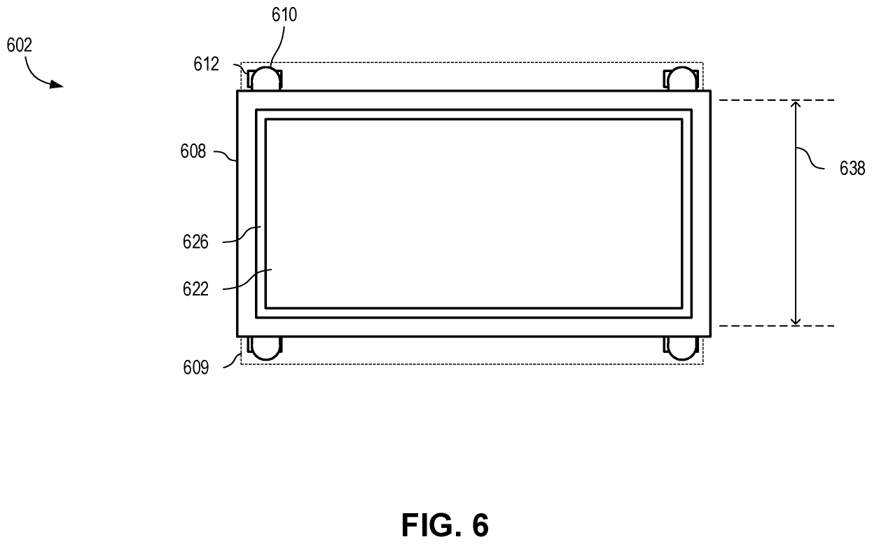

FIG. 6 is a top schematic view of a patient transfer assembly 602, according to certain aspects of the present disclosure. Patient transfer assembly 602 can be any suitable patient transfer assembly, such as patient transfer assembly 102 of FIG. 1. The patient transfer assembly 602 includes a support frame 608 supporting walls. Legs 610 can couple the support frame 608 to rollers 612. The rollers 612 can define a base 609. As depicted in FIG. 6, the support frame 608 is supported by four legs 610, each adjacent respective corners of the rectangular support frame 608. Any number of legs 610 can be used.

The support frame 608 can have a length (e.g., left-to-right as seen in FIG. 6) and a width (e.g., up-to-down as seen in FIG. 6). The legs 610 can be spaced apart in the width direction by a distance 638. This distance 638 can be greater than the width of the destination n surface (e.g., imaging table), thus permitting the patient transfer assembly 602 to be easily rolled over destination surface. Likewise, in examples where the receiving space is bounded by the support frame 608, the receiving space and any relevant openings can have widths that are greater than the width of the destination surface.

A support surface 622 can be coupled to the support frame 608 via an intermediary support 626, although in some cases the support surface 622 can be otherwise directly coupled to the support frame 608. The intermediary support 626 can be a region of material that is coupled to the support frame 608 and includes coupling features for coupling to the support surface 622. For example, in some cases, the intermediary support 626 can include a zipper portion designed to engage a zipper portion of the support surface 622. Any suitable detachable coupling feature can be used. In some cases, intermediary support 626 can coupled to support surface 622 over the entire perimeter of the support surface 622.

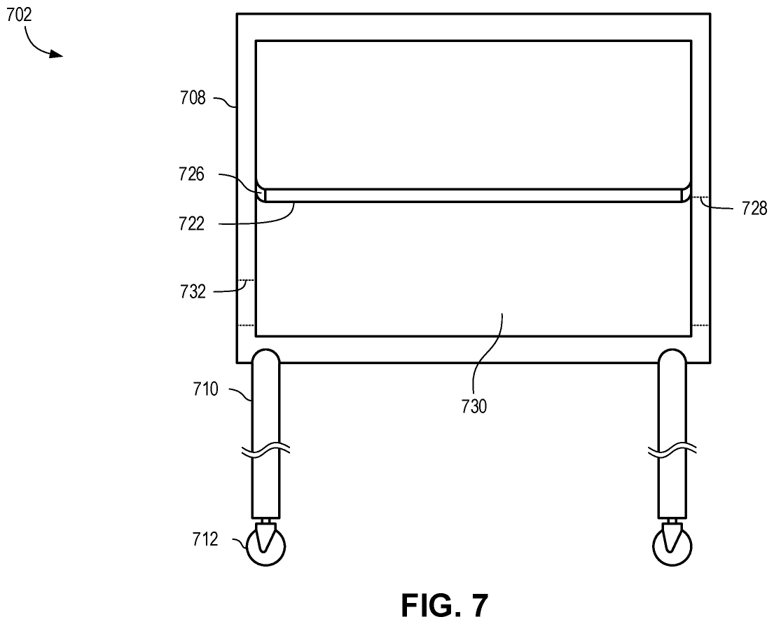

FIG. 7 is a side schematic view of a patient transfer assembly 702, according to certain aspects of the present disclosure. Patient transfer assembly 702 can be any suitable patient transfer assembly, such as patient transfer assembly 602 of FIG. 6. For illustrative purposes, the walls are not depicted. Support frame 708 is depicted supporting support surface 722 via an intermediary support 726. The support frame 708 is supported by legs 710 coupled to rollers 712. As depicted in FIG. 7, the support surface 722 can be in a raised position.

As depicted in FIG. 7, the receiving space 730 is enclosed by the support frame 708, although that need not always be the case. In such a case, a front opening 728 can permit a destination surface (e.g., an imaging table) to be moved into the receiving space 730. In some cases, an additional rear opening 732 can be used, thus ensuring the patient is placed on the destination surface at location further from the end of the destination surface than if no rear opening 732 were present. In some cases, the front opening 728 can be sized to remain below a top of the support surface 722 when the support surface 722 is in the raised position. The front opening 728 can be sized to permit the patient transfer assembly 702 to be easily removed from the destination surface without disturbing a patient sleeping on the destination surface. The rear opening 732, however, does not need to be large enough to pass the destination surface and a patient therethrough. In some cases, the height of the rear opening 732 is approximately the thickness of the destination surface, or slightly larger. The height of the rear opening 732 can be smaller than the height of the front opening 728.

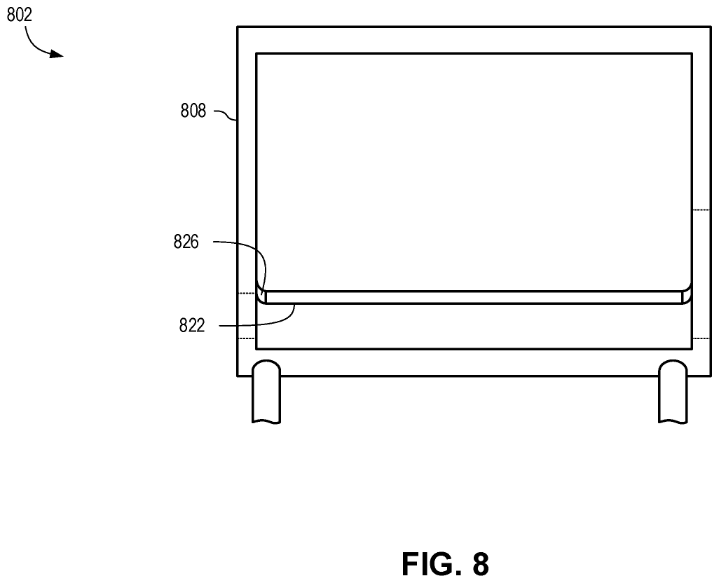

FIG. 8 is a side schematic view of a patient transfer assembly 802 with the support surface 822 lowered, according to certain aspects of the present disclosure. Patient transfer assembly 802 can be any suitable patient transfer assembly, such as patient transfer assembly 702 of FIG. 7, after lowering of the support surface. For illustrative purposes, the walls are not depicted. The support surface 822 can be lowered (e.g., moved to the deployed position) by lowering the intermediary support 826, such as disclosed herein. The intermediary support 826 can be movable between an intermediary support transport position (e.g., a raised position) and an intermediary support deployed position (e.g., a lowered position).

In some cases, the intermediary support 826 can be coupled to the support frame 808 using one or more reels. The reels can be used to raise and/or lower the intermediary support 826.

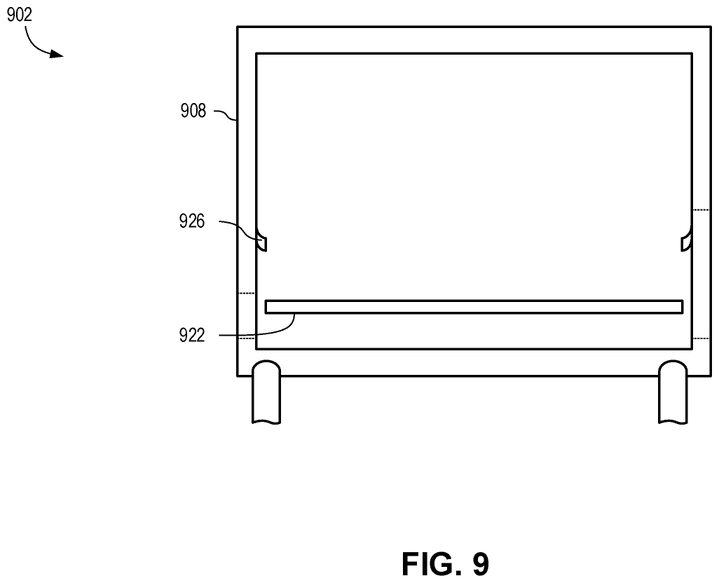

FIG. 9 is a side schematic view of a patient transfer assembly 902 with the support surface 922 detached, according to certain aspects of the present disclosure. Patient transfer assembly 902 can be any suitable patient transfer assembly, such as patient transfer assembly 802 of FIG. 8, after detaching of the support surface 922. For illustrative purposes, the walls are not depicted. The support surface 922 can be detached from the support frame 908 by detaching the support surface 922 from intermediary support 926.

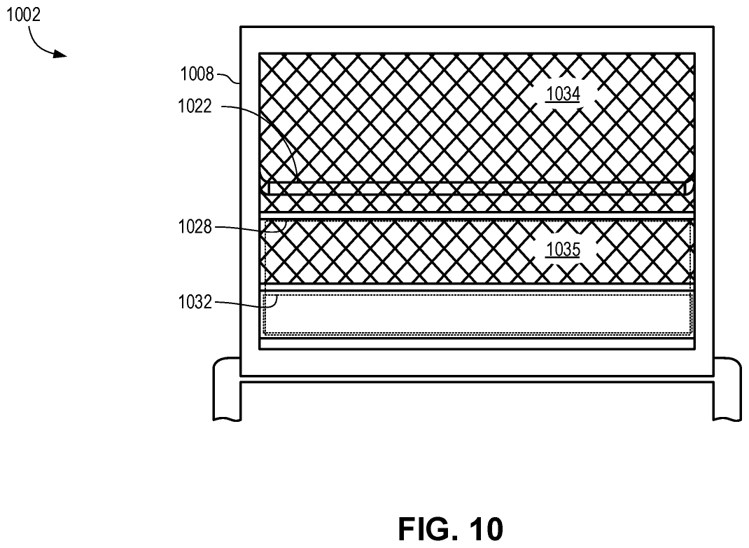

FIG. 10 is a front schematic view of a patient transfer assembly 1002, according to certain aspects of the present disclosure. Patient transfer assembly 1002 can be any suitable patient transfer assembly, such as patient transfer assembly 602 of FIG. 6. The patient transfer assembly 1002 can include a support frame 1008 supporting a front wall 1034 and a rear wall 1035, as well as a support surface 1022.

For illustrative purposes, front opening 1028 and rear opening 1032 are each indicated by respective approximated dotted rectangles. The front wall 1034 is visible at the front of the patient transfer assembly 1002 (e.g., out of the page towards the viewer as depicted in FIG. 10), while the opposing rear wall 1035 is visible through the front opening 1028.

The rear opening 1032 is sized to accept a destination surface (e.g., an imaging table). In some cases, the rear opening 1032 is sized to accept a destination surface, but prevent passage of both a destination surface and a patient.

The front opening 1028 is sized to permit passage of a destination surface (e.g., imaging table), a support surface 1022, and a patient (e.g., a young patient, such as a patient below 0.5, 1, 1.5, or 2 years of age) thereon.

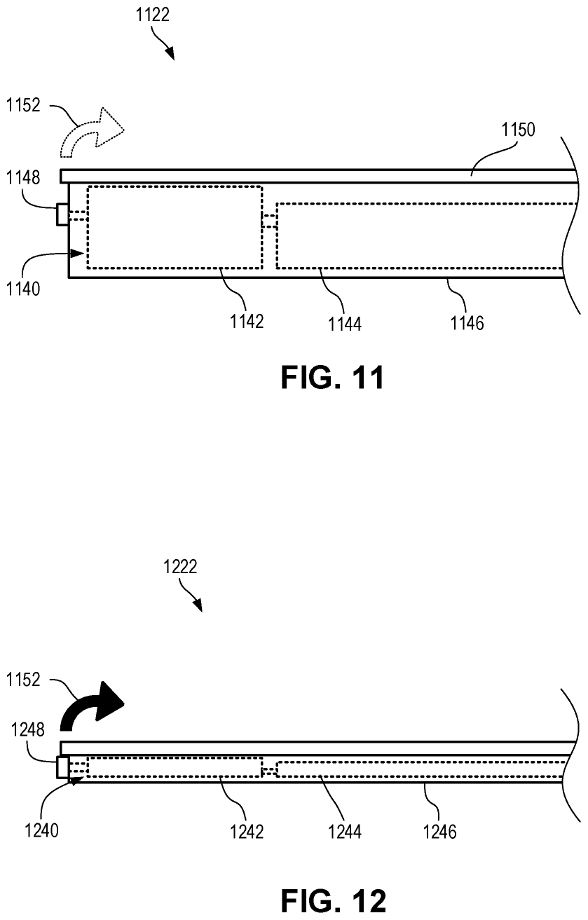

FIG. 11 is a side schematic view of a portion of a support surface 1122 with an inflated stiffening support 1140, according to certain aspects of the present disclosure. Support surface 1122 can be support surface 122 of FIG. 1. The support surface 1122 can include main body 1146 made of a flexible material and an attachment mechanism 1150 (e.g., a zipper). The support surface 1122 can include a stiffening support 1140, which can include any number of inflatable bladders. As depicted in FIG. 11, a first bladder 1142 and a second bladder 1144 are shown, although in some cases the support surface 1122 can include only a single bladder, and in some cases the support surface 1122 can include additional bladders (e.g., a set of bladders located behind the first bladder 1142 and second bladder 1144).

First bladder 1142 can be coupled to second bladder 1144 via a fluid channel. In some cases, first bladder 1142 can be positioned between an edge of the support surface 1122 and the second bladder 1144, and can be taller (e.g., in the up-down direction as depicted in FIG. 11) than second bladder 1144, which can facilitate urging a patient away from edges of the support surface 1122 (e.g., towards a center of the support surface 1122).

A valve 1148 can be coupled to the stiffening support 1140, such a as to first bladder 1142. The valve 1148 can permit pressurized fluid (e.g., air) to enter the first bladder 1142 and second bladder 1144 from a source of pressurized fluid (e.g., an air compressor), but can manually or automatically seal to prevent the fluid from leaking out into the surrounding environment when the source of pressurized fluid is removed. In some cases, valve 1148 can be actuated to deflate the stiffening support 1140, thus permitting pressurized fluid within the first bladder 1142 and second bladder 1144 to exit into the surrounding environment.

When the stiffening support 1140 is inflated, the support surface 1122 can be considered to be in an inflated configuration. In the inflated configuration, the stiffening support 1140 can resist bending or flexion of the main body 1146 of support surface 1122 (e.g., in direction 1152).

FIG. 12 is a side schematic view of a portion of a support surface 1222 with a deflated stiffening support 1240, according to certain aspects of the present disclosure. Support surface 1222 can be support surface 1122 of FIG. 11 after deflation of its stiffening support 1140.

Deflation of the stiffening support 1240 can be accomplished by actuating valve 1248 to permit fluid (e.g., air) to escape into the surrounding environment from first bladder 1242 and second bladder 1244.

When the stiffening support 1240 is deflated, the support surface 1222 can be considered to be in a deflated configuration. In the deflated configuration, the stiffening support 1240 can permit bending or flexion of the main body 1246 of support surface 1222 (e.g., in direction 1252).

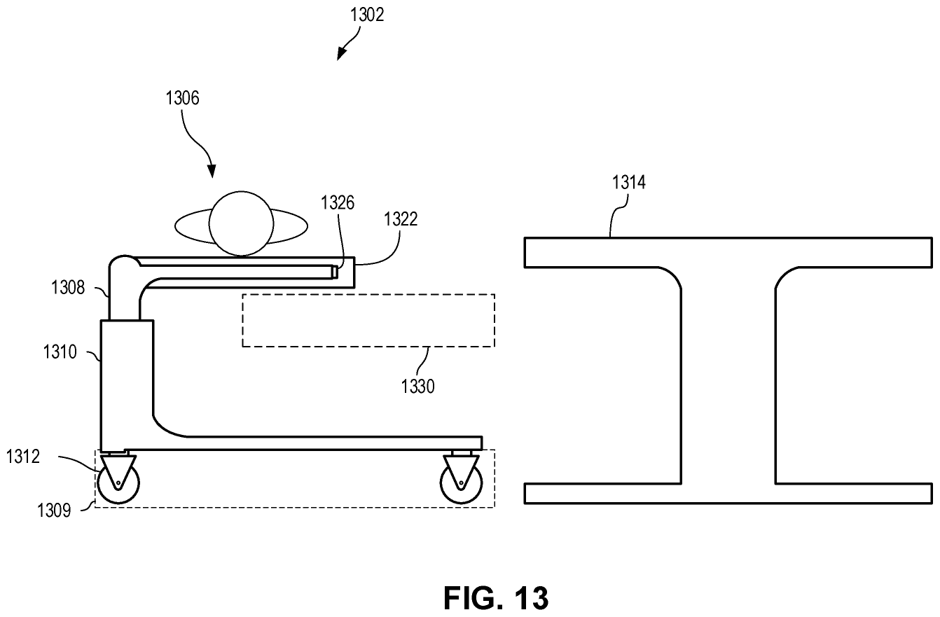

FIG. 13 is a side schematic diagram depicting a patient transfer assembly 1302 positioned adjacent a destination surface 1314, according to certain aspects of the present disclosure. The patient transfer assembly 1302 is depicted in a bed-like form, although that need not always be the case. The destination surface 1314 is depicted in a table-like form (e.g., an operating table), although that need not always be the case.

The patient transfer assembly 1302 includes a support frame 1308 that supports a support surface 1322 via an intermediary support 1326. A patient 1306 can rest upon the support surface 1322. The support surface 1322 is in a transport position. The transport position can be a neutral position suitable for transporting the patient.

The support frame 1308 is coupled to a set of rollers 1312 that define a base 1309. As depicted, the support frame 1308 is coupled to the set of rollers 1312 via leg(s) 1310. In some cases, the leg(s) 1310 and/or support frame 1308 can be telescoping (e.g., individually or together), such as to adjust the vertical distance of the support frame 1308 from the base 1309 (e.g., to adjust the vertical distance of the support surface 1322 above the floor).

The patient transfer assembly 1302 includes a receiving space 1330 that is located adjacent the support surface 1322. More specifically, the receiving space 1330 is located horizontally offset and slightly below the support surface 1322.

The patient transfer assembly 1302 is depicted spaced apart from the destination surface 1314, ready to approach the destination surface 1314. As depicted in FIG. 13, the support frame 1308 may be raised (e.g., via a linear actuator) prior to the patient transfer assembly 1302 approaching the destination surface 1314 such that the support surface 1322 can pass over the destination surface 1314.

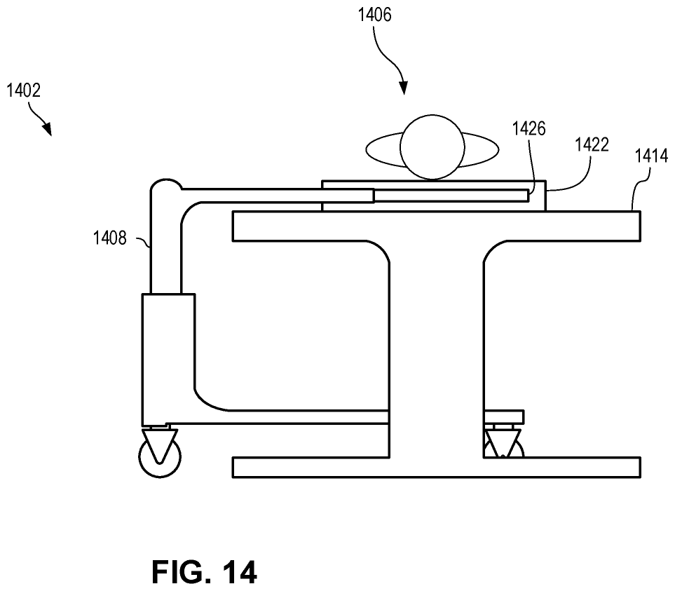

FIG. 14 is a side schematic diagram depicting a patient transfer assembly 1402 with its support surface 1422 extended over a destination surface 1414, according to certain aspects of the present disclosure. Patient transfer assembly 1402 can be any suitable patient transfer assembly, such as patient transfer assembly 1302 of FIG. 13 after its support frame has been raised, it has been rolled towards the destination surface, and its support surface moved into a deployed position.

The support surface 1422 of the patient transfer assembly 1402 is in a deployed position. In the deployed position, the support surface 1422 is ready to be separated from the support frame 1408. As depicted in FIG. 14, the support surface 1422 is removably coupled to an intermediary support 4126, which is in turn coupled to the frame 1408. The intermediary support 4126 can include an optional frame coupled to one or more slides (e.g., drawer slides), which facilitates moving the support surface 4122 in a horizontal direction (e.g., parallel to the floor).

As depicted in FIG. 14, the support surface 1422 includes inflatable stiffening support(s) in an inflated configuration.

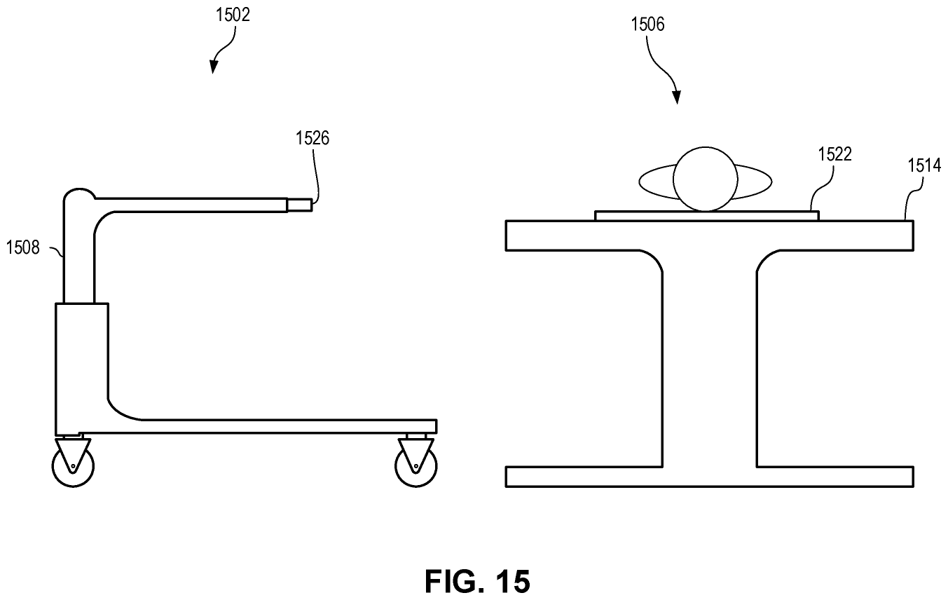

FIG. 15 is a side schematic diagram depicting a patient transfer assembly 1502 spaced apart from a destination surface 1514 with its support surface 1522 separated and remaining on the destination surface 1514, according to certain aspects of the present disclosure. Patient transfer assembly 1502 can be any suitable patient transfer assembly, such as patient transfer assembly 1402 of FIG. 14 after its support surface is detached.

The support surface 1522 is separated from the support frame 1508, having been detached from the intermediary support 1526. The intermediary support 1526 is moved (e.g., horizontally slid) back towards a transport position. Other than the support surface 1522, the remainder of the patient transfer assembly 1502 has been rolled away from the destination surface 1514.

The support surface 1522 remains on the destination surface 1514, supporting the patient 1506. The support surface 1522 is depicted in a deflated configuration. In the deflated configuration, the patient 1506 can be supported closer to the destination surface 1514, and the support surface 1522 itself can be more flexible than when in an inflated configuration.

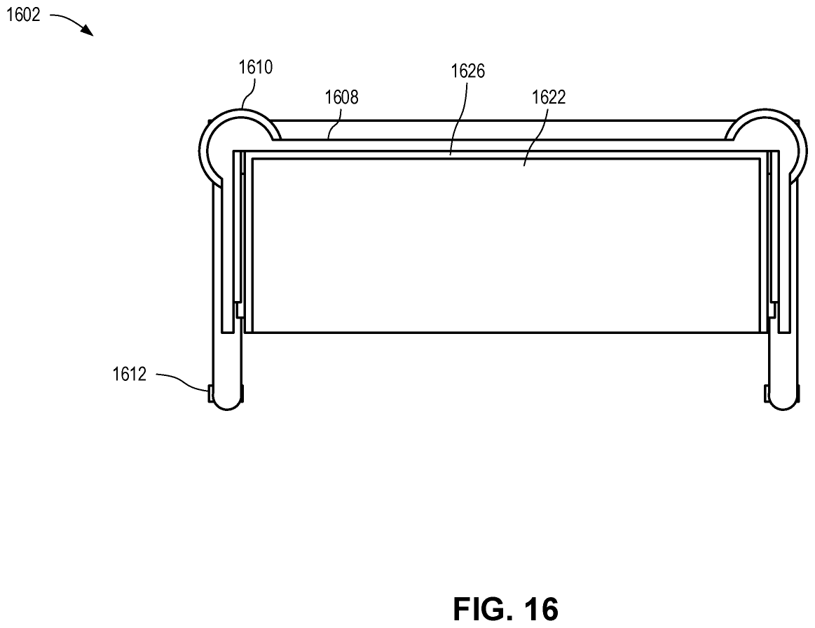

FIG. 16 is an overhead schematic diagram depicting a patient transfer assembly 1602 with its support surface 1622 in a transport position, according to certain aspects of the present disclosure. The patient transfer assembly 1602 can be any suitable patient transfer assembly, such as patient transfer assembly 1302 of FIG. 13.

The support frame 1608 is supported by rollers 1612 via legs 1610. As depicted in FIG. 16, two legs 1610 are shown. The support surface 1622 is in a transport position (e.g., a retracted position). The support surface 1622 is coupled to the support frame 1608 via an intermediary support 1626. The intermediary support 1626 takes the form of a sub-frame that is slidably coupled to the support frame 1608 via two slides (e.g., drawer slides). Thus, the intermediary support 1626 is movable between an intermediary support transport (e.g., retracted) position and an intermediary support deployed (e.g., extended) position. When the intermediary support 1626 is in the intermediary support transport (e.g., retracted) position, the attached support surface 1622 is in the transport position. When the intermediary support 1626 is in the intermediary support deployed (e.g., extended) position, the attached support surface 1622 would be in the deployed position.

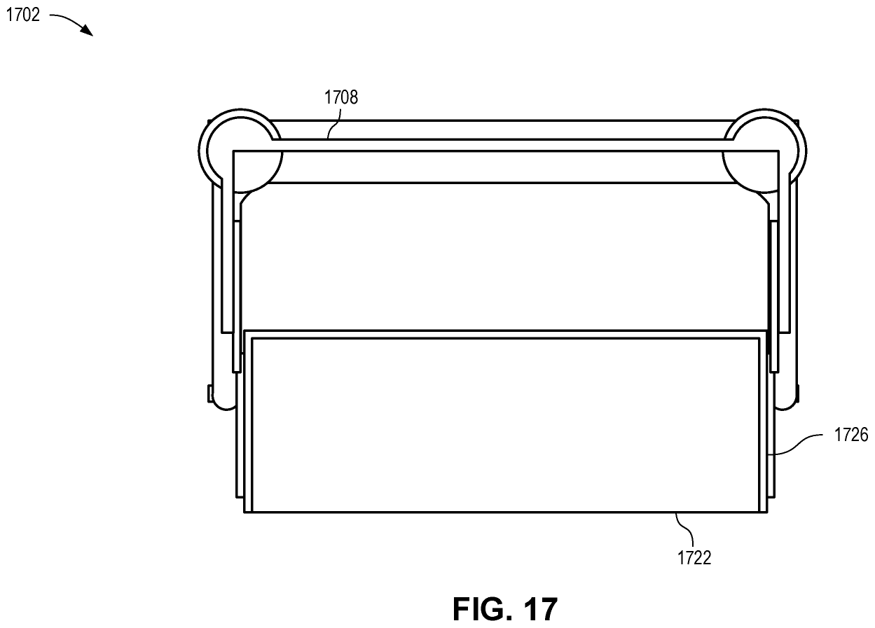

FIG. 17 is an overhead schematic diagram depicting a patient transfer assembly 1702 with its support surface 1722 in a deployed position, according to certain aspects of the present disclosure. The patient transfer assembly 1702 can be any suitable patient transfer assembly, such as patient transfer assembly 1602 of FIG. 16 after its support surface is moved to the deployed position.

The intermediary support 1726 is shown as being in its intermediary support deployed position. In this position, the intermediary support 1726 has been extended away from the support frame 1708 (e.g., horizontally displaced with respect to the base defined by the rollers). Thus, the attached support surface 1722 has been moved (e.g., horizontally displaced with respect to the base defined by the rollers) into its deployed position.

In this deployed position, the support surface 1722 is ready to be detached from the intermediary support 1726, and thus separated from the support frame 1708.

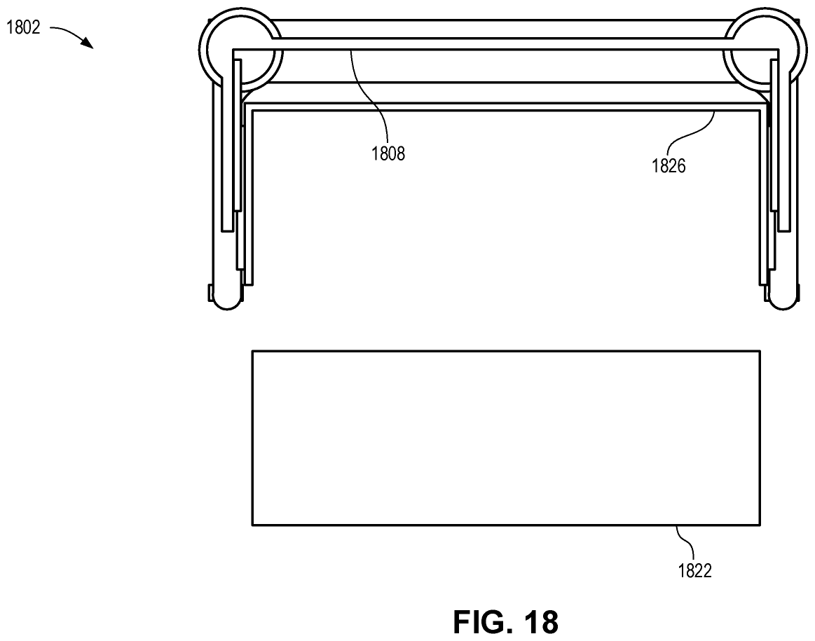

FIG. 18 is an overhead schematic diagram depicting a patient transfer assembly 1802 with its support surface 1822 separated from the support frame 1808, according to certain aspects of the present disclosure. The patient transfer assembly 1802 can be any suitable patient transfer assembly, such as patient transfer assembly 1702 of FIG. 17 after its support surface has been detached.

The support surface 1822 has been detached from the intermediary support 1826, and thus separated from the support frame 1808. Once separated, the support surface 1822 can remain in place (e.g., on a destination surface) while the remainder of the patient transfer assembly 1802 is moved away. Additionally, the intermediary support 1826 can optionally be moved back to its intermediary support transport position.

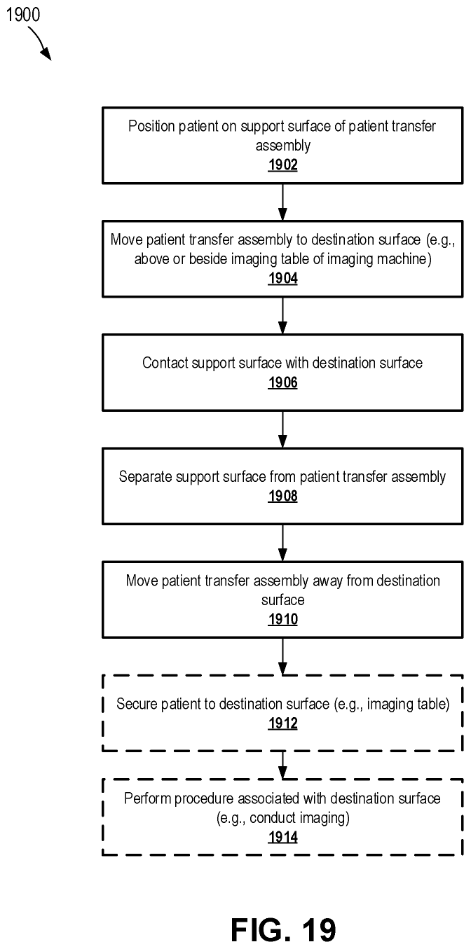

FIG. 19 is a flowchart depicting a process 1900 for using a patient transfer assembly, according to certain aspects of the present disclosure. The process 1900 can be used in conjunction with patient transfer assembly 102 and imaging machine 104 of FIG. 1, or any other suitable patient transfer assembly and destination surface.

At block 1902, the patient is positioned on the support surface of the patient transfer assembly. In some cases, positioning the patient on the support surface includes allowing the patient to fall asleep in the patient transfer assembly (e.g., on the support surface). For example, the patient can be a young child, baby, or infant, such as one under the age of 2, 1.5, 1, or 0.5 years of age. The patient can be permitted to fall asleep in the patient transfer assembly when the patient transfer assembly is in a different room than the imaging machine. For example, the patient transfer assembly can be placed in a room conducive to falling asleep (e.g., a darkened and/or quiet room) while the patient is allowed to fall asleep at block 1902. In some cases, a stiffening support of the support surface can be engaged, such as by inflation (e.g., inflation of a set of one or more bladders). Inflation of a stiffening support can occur in a room away from the imaging machine.

At block 1904, the patient transfer assembly can be moved to the destination surface, such that the destination surface is within the receiving space of the patient transfer assembly. In an example, the support surface of the patient transfer assembly can be moved above and/or beside an imaging table, which can include moving the patient transfer assembly such that a portion of the imaging table is positioned within the receiving space of the patient transfer assembly (e.g., a space below or adjacent to the support surface of the patient transfer assembly, optionally within the support frame of the patient transfer assembly). In some cases, moving the patient transfer assembly to the destination surface can include moving the patient transfer assembly into the same room as the destination surface. In some cases, moving the patient transfer assembly to the destination surface can include securing the patient transfer assembly to the destination surface, such as to a base of an imaging table.

At block 1906, the support surface of the patient transfer assembly can be contacted with the destination surface. Contacting the support surface can include moving the support surface with respect to the destination surface until the support surface rests upon the destination surface. Contacting the support surface can include moving the support surface with respect to the base (e.g., base defined by the rollers of the patient transfer assembly). Contacting the support surface can include moving the support surface from a transport position to a deployed position.

Contacting the support surface with the destination surface can include i) lowering the support surface until the support surface rests on the destination surface; ii) horizontally displacing the support surface with respect to the base; iii) raising the destination surface until the support surface rests on the destination surface; or iv) any combination of i-iii. Contacting the support surface with the destination surface can include allowing the weight of the patient to be fully supported by the destination surface.

At block 1908, the support surface can be separated from the patient transfer assembly. Separating, or detaching, the support surface from the patient transfer assembly can include separating the support surface from an intermediary support. In an example, separating the support surface from the patient transfer assembly can include unzipping a zipper surrounding a perimeter of the support surface. In some cases, if a stiffening support is used, the stiffening support can be disengaged at any of block 1908, block 1910, or block 1912. Disengaging the stiffening support can include deflating a set of one or more inflatable bladders.

At block 1910, the patient transfer assembly can be moved away from the destination surface. In some cases, moving the patient transfer assembly away from the destination surface can include adjusting a height of the patient transfer assembly and/or adjusting a height of the destination surface to facilitate moving the patient transfer assembly away from the destination surface without contacting the patient. The patient transfer assembly can be moved away from the destination surface, leaving the patient and support surface resting on the destination surface. As used herein with reference to block 1910, and as appropriate herein, moving the patient transfer assembly away from the destination surface can mean moving the remainder of the patient transfer assembly, other than the support surface, away from the destination surface. In some cases, block 1910 can be skipped, and instead of moving the patient transfer assembly away from the destination surface, the support surface can be slid along the destination surface to position the patient in a desired position (e.g., the correct position for imaging on an imaging table of an imaging machine).

At optional block 1912, the patient can be secured to the destination surface (e.g., an imaging table). In some cases, securing the patient to the destination surface can include securing the patient to the destination surface with the support surface positioned between the patient and the destination surface. In some cases, however, the support surface can be removed prior to securing the patient. In some cases, if a stiffening support is used, the stiffening support can be sufficiently disengaged (e.g., the set of bladders can be sufficiently deflated) to permit the support surface to be flexed and maneuvered as desired, such as into position within a piece of imaging equipment (e.g., a removable piece of imaging equipment), such as an MR imaging coil.

At optional block 1914, one or more procedures associated with the destination can be performed. For example, if the destination surface is an imaging table of an imaging machine, imaging can be conducted. Conducting imaging can include performing any suitable imaging procedures, such as one or more MR scans. In another example, if the destination surface is a surgical table, a surgical procedure can be performed. In some cases, procedures, such as imaging or surgical procedures, can be conducted without removal of the support surface from beneath the patient.

After a procedure is performed (e.g., after imaging is conducted), the patient can be returned to the patient transfer assembly or otherwise removed from the destination surface. For example, when a patient transfer assembly is used to ensure a sleeping patient does not awaken before an imaging procedure, the patient may be simply awakened after the imaging procedure is completed. Likewise, whenever it is desired to move the patient again, the patient can be returned to the same or a different, but still compatible, patient transfer assembly. If returning to a patient transfer assembly, the patient transfer assembly can be moved into position over the destination surface, such as in block 1904, then the support surface can be reattached to the support frame of the patient transfer assembly, such as the opposite of block 1908, then the support surface can be moved with respect to the destination surface to separate the support surface and the destination surface, then the patient transfer assembly can be moved away from the destination with the patient. If a stiffening support is used, the stiffening support can be re-engaged (e.g., the set of bladders inflated) prior to separating the support surface and the destination surface.

The foregoing description of the embodiments, including illustrated embodiments, has been presented only for the purpose of illustration and description and is not intended to be exhaustive or limiting to the precise forms disclosed. Numerous modifications, adaptations, and uses thereof will be apparent to those skilled in the art. Numerous changes to the disclosed embodiments can be made in accordance with the disclosure herein, without departing from the spirit or scope of the invention. Thus, the breadth and scope of the present invention should not be limited by any of the above described embodiments.

Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

As used below, any reference to a series of examples is to be understood as a reference to each of those examples disjunctively (e.g., “Examples 1-4” is to be understood as “Examples 1, 2, 3, or 4”).

Example 1 is a patient transfer assembly, comprising: a frame supporting a set of walls; a support surface removably coupled to the frame to support a patient, the support surface movable between a raised position and a lowered position, wherein the support surface is detachable from the frame when in the lowered position; and a receiving space located below the support surface for receiving a portion of an imaging table, wherein the support surface rests on the imaging table when in the lowered position.

Example 2 is the patient transfer assembly of example(s) 1, further comprising: a set of rollers coupled to the frame by one or more legs to facilitate movement of the frame with respect to the imaging table.

Example 3 is the patient transfer assembly of example(s) 1 or 2, wherein the receiving space is bounded at least in part by the frame.

Example 4 is the patient transfer assembly of example(s) 1-3, wherein support surface includes a set of one or more inflatable bladders, wherein the support surface is transitionable between an inflated configuration and a deflated configuration, wherein the support surface resists flexion in the inflated configuration, and wherein the support surface permits flexion in the deflated configuration.

Example 5 is the patient transfer assembly of example(s) 1-4, wherein the support surface is removably coupled to the frame using at least one selected from the group consisting of a zipper, a hook and loop fastener, and a hook.

Example 6 is the patient transfer assembly of example(s) 1-5, wherein the support surface is removably coupled to an intermediary support, wherein the intermediary support is coupled to the frame and movable between a raised position and a lowered position.

Example 7 is the patient transfer assembly of example(s) 1-6, wherein the support surface is made from one or more non-ferromagnetic materials.

Example 8 is the patient transfer assembly of example(s) 1-7, wherein the frame is made from one or more non-ferromagnetic materials.

Example 9 is the patient transfer assembly of example(s) 1-8, wherein the support surface is made from material radiolucent under magnetic resonance imaging.

Example 10 is the patient transfer assembly of example(s) 1-9, wherein the frame has a width and a length, wherein the receiving space is configured to receive the portion of the imaging table in a direction parallel the length of the frame, wherein the width of the frame is greater than a width of the imaging table.

Example 11 is a method, comprising: providing a patient transfer assembly for use with an imaging table of an imaging device, wherein the patient transfer assembly comprises: a frame supporting a set of walls; a support surface removably coupled to the frame to support a patient, the support surface movable between a raised position and a lowered position, wherein the support surface is detachable from the frame when in the lowered position; and a receiving space located below the support surface for receiving a portion of the imaging table, wherein the support surface rests on the imaging table when in the lowered position; maneuvering the patient transfer assembly towards the imaging device to position the imaging table within the receiving space below the support surface; lowering the support surface so that the support surface rests upon the imaging table; detaching the support surface from the frame; and maneuvering the frame away from the imaging device.

Example 12 is the method of example(s) 11, wherein maneuvering the patient transfer assembly comprises rolling the patient transfer assembly on a set of rollers coupled to the frame by one or more legs.

Example 13 is the method of example(s) 11 or 12, wherein the receiving space is bounded at least in part by the frame.

Example 14 is the method of example(s) 11-13, wherein maneuvering the patient transfer assembly towards the imaging device comprises moving the patient transfer assembly from a position outside of a room containing the imaging device into the room containing the imaging device.

Example 15 is the method of example(s) 11-14, wherein the support surface is removably coupled to the frame using at least one selected from the group consisting of a zipper, a hook and loop fastener, and a hook.

Example 16 is the method of example(s) 11-15, wherein detaching the support surface from the frame comprises detaching the support surface from an intermediary support coupled to the frame, and wherein lowering the support surface comprises lowering the intermediary support.

Example 17 is the method of example(s) 11-16, wherein the support surface is made from one or more non-ferromagnetic materials.

Example 18 is the method of example(s) 11-17, wherein the support surface is made from material transparent to magnetic resonance imaging.

Example 19 is the method of example(s) 11-18, wherein the frame is made from one or more non-ferromagnetic materials.

Example 20 is the method of example(s) 11-19, wherein the frame has a width and a length, wherein the receiving space is configured to receive the portion of the imaging table in a direction parallel the length of the frame, wherein the width of the frame is greater than a width of the imaging table.

Example 21 is the method of example(s) 11-20, wherein the support surface includes a set of one or more inflatable bladders, wherein the support surface is transitionable between an inflated configuration and a deflated configuration, wherein the support surface resists flexion in the inflated configuration, and wherein the support surface permits flexion in the deflated configuration; the method further comprising: inflating the set of inflatable bladders, prior to maneuvering the patient transfer assembly towards the imaging device, to transition the support surface into the inflated configuration; and deflating the set of inflatable bladders, after lowering the support surface, to transition the support surface into the deflated configuration.

Example 22 is a patient transfer assembly, comprising: a support frame; a set of rollers defining a base, the set of rollers coupled to the support frame to facilitate movement of the support frame; and a support surface removably coupled to the support frame to support a patient, the support surface movable with respect to the base between a transport position and a deployed position, wherein the support surface is detachable from the support frame when in the deployed position.

Example 23 is the patient transfer assembly of example(s) 22, further comprising a receiving space for receiving a destination surface, wherein the receiving space is located below or adjacent to the support surface when the support surface is in the transport position, wherein the support surface rests on the destination surface when in the deployed position.

Example 24 is the patient transfer assembly of example(s) 22 or 23, further comprising a set of walls coupled to the support frame, wherein the support surface is at least partially enclosed by the set of walls when in the transport position.

Example 25 is the patient transfer assembly of example(s) 24, wherein the receiving space is bounded at least in part by the set of walls.

Example 26 is the patient transfer assembly of example(s) 22-25, wherein the support frame has a width and a length, wherein the receiving space is configured to receive a portion of a destination surface in a direction parallel the length of the support frame, wherein the width of the support frame is greater than a width of the destination surface.

Example 27 is the patient transfer assembly of example(s) 22-26, wherein the support surface includes a set of one or more inflatable bladders, wherein the support surface is transitionable between an inflated configuration and a deflated configuration, wherein the support surface resists flexion in the inflated configuration, and wherein the support surface permits flexion in the deflated configuration.

Example 28 is the patient transfer assembly of example(s) 22-27, wherein the support surface is removably coupled to the support frame using at least one selected from the group consisting of a zipper, a hook and loop fastener, and a hook.

Example 29 is the patient transfer assembly of example(s) 22-28, wherein the support surface is removably coupled to an intermediary support, wherein the intermediary support is coupled to the support frame and movable between a transport position and a deployed position.

Example 30 is the patient transfer assembly of example(s) 22-29, wherein the support surface is non-ferromagnetic and radiolucent.

Example 31 is the patient transfer assembly of example(s) 22-30, wherein the support frame is non-ferromagnetic.

Example 32 is the patient transfer assembly of example(s) 22-31, wherein the support surface is coupled to the support frame for vertical movement such that movement of the support surface from the transport position to the deployed position includes vertically lowering the support surface towards the base.

Example 33 is the patient transfer assembly of example(s) 22-32, wherein the support surface is coupled to the support frame for horizontal movement such that movement of the support surface from the transport position to the deployed position includes horizontally displacing the support surface with respect to the base.

Example 34 is a method, comprising: providing a patient transfer assembly for use with a destination surface, wherein the patient transfer assembly comprises: a support frame; a set of rollers defining a base, the set of rollers coupled to the support frame to facilitate movement of the support frame; a support surface removably coupled to the support frame to support a patient, the support surface movable with respect to the base between a transport position and a deployed position, wherein the support surface is detachable from the support frame when in the deployed position; and a receiving space for receiving the destination surface, wherein the receiving space is located below or adjacent to the support surface when the support surface is in the transport position, and wherein the support surface rests on the destination surface when in the deployed position; maneuvering the patient transfer assembly towards the destination surface to position the destination surface within the receiving space of the patient transfer assembly; moving the support surface from the transport position to the deployed position such that the support surface rests upon the destination surface; detaching the support surface from the support frame; and maneuvering the support frame away from the destination surface.

Example 35 is the method of example(s) 34, wherein the patient transfer assembly further comprises a set of walls coupled to the support frame, wherein the support surface is at least partially enclosed by the set of walls when in the transport position, and wherein the receiving space is bounded at least in part by the set of walls.

Example 36 is the method of example(s) 34 or 35, wherein the support frame has a width and a length, wherein the receiving space is configured to receive a portion of the destination surface in a direction parallel the length of the support frame, wherein the width of the support frame is greater than a width of the destination surface.

Example 37 is the method of example(s) 34-36, wherein the support surface is removably coupled to the support frame using at least one selected from the group consisting of a zipper, a hook and loop fastener, and a hook.

Example 38 is the method of example(s) 34-37, wherein detaching the support surface from the support frame comprises detaching the support surface from an intermediary support coupled to the support frame, and wherein moving the support surface from the transport position to the deployed position comprises moving the intermediary support form an intermediary support transport position to an intermediary support deployed position.

Example 39 is the method of example(s) 34-38, wherein the support surface includes a set of one or more inflatable bladders, wherein the support surface is transitionable between an inflated configuration and a deflated configuration, wherein the support surface resists flexion in the inflated configuration, and wherein the support surface permits flexion in the deflated configuration; the method further comprising: inflating the set of inflatable bladders, prior to maneuvering the patient transfer assembly towards the destination surface, to transition the support surface into the inflated configuration; and deflating the set of inflatable bladders, after moving the support surface to the deployed position, to transition the support surface into the deflated configuration.

Example 40 is the method of example(s) 34-39, wherein moving the support surface from the transport position to the deployed position includes vertically lowering the support surface onto the destination surface.

Example 41 is the method of example(s) 34-40, wherein moving the support surface from the transport position to the deployed position includes horizontally displacing the support surface with respect to the base

What is claimed is:

1. A patient transfer assembly, comprising:
a support frame;
a set of rollers defining a base, the set of rollers coupled to the support frame to facilitate movement of the support frame; and
a support surface removably coupled to the support frame to support a patient, the support surface movable with respect to the base between a transport position and a deployed position, wherein the support surface is detachable from the support frame when in the deployed position.

2. The patient transfer assembly of claim 1, further comprising a receiving space for receiving a destination surface, wherein the receiving space is located below or adjacent to the support surface when the support surface is in the transport position, wherein the support surface rests on the destination surface when in the deployed position.

3. The patient transfer assembly of claim 1, further comprising a set of walls coupled to the support frame, wherein the support surface is at least partially enclosed by the set of walls when in the transport position.

4. The patient transfer assembly of claim 3, wherein the receiving space is bounded at least in part by the set of walls.

5. The patient transfer assembly of claim 1, wherein the support frame has a width and a length, wherein the receiving space is configured to receive a portion of a destination surface in a direction parallel the length of the support frame, wherein the width of the support frame is greater than a width of the destination surface.

6. The patient transfer assembly of claim 1, wherein the support surface includes a set of one or more inflatable bladders, wherein the support surface is transitionable between an inflated configuration and a deflated configuration, wherein the support surface resists flexion in the inflated configuration, and wherein the support surface permits flexion in the deflated configuration.

7. The patient transfer assembly of claim 1, wherein the support surface is removably coupled to the support frame using at least one selected from the group consisting of a zipper, a hook and loop fastener, and a hook.

8. The patient transfer assembly of claim 1, wherein the support surface is removably coupled to an intermediary support, wherein the intermediary support is coupled to the support frame and movable between a transport position and a deployed position.

9. The patient transfer assembly of claim 1, wherein the support surface is non-ferromagnetic and radiolucent.

10. The patient transfer assembly of claim 7, wherein the support frame is non-ferromagnetic.

11. The patient transfer assembly of claim 1, wherein the support surface is coupled to the support frame for vertical movement such that movement of the support surface from the transport position to the deployed position includes vertically lowering the support surface towards the base.

12. The patient transfer assembly of claim 1, wherein the support surface is coupled to the support frame for horizontal movement such that movement of the support surface from the transport position to the deployed position includes horizontally displacing the support surface with respect to the base.

13. A method, comprising:
providing a patient transfer assembly for use with a destination surface, wherein the patient transfer assembly comprises:
a support frame;
a set of rollers defining a base, the set of rollers coupled to the support frame to facilitate movement of the support frame;
a support surface removably coupled to the support frame to support a patient, the support surface movable with respect to the base between a transport position and a deployed position, wherein the support surface is detachable from the support frame when in the deployed position; and
a receiving space for receiving the destination surface, wherein the receiving space is located below or adjacent to the support surface when the support surface is in the transport position, and wherein the support surface rests on the destination surface when in the deployed position;
maneuvering the patient transfer assembly towards the destination surface to position the destination surface within the receiving space of the patient transfer assembly;
moving the support surface from the transport position to the deployed position such that the support surface rests upon the destination surface;
detaching the support surface from the support frame; and
maneuvering the support frame away from the destination surface.

14. The method of claim 13, wherein the patient transfer assembly further comprises a set of walls coupled to the support frame, wherein the support surface is at least partially enclosed by the set of walls when in the transport position, and wherein the receiving space is bounded at least in part by the set of walls.

15. The method of claim 13, wherein the support frame has a width and a length, wherein the receiving space is configured to receive a portion of the destination surface in a direction parallel the length of the support frame, wherein the width of the support frame is greater than a width of the destination surface.

16. The method of claim 13, wherein the support surface is removably coupled to the support frame using at least one selected from the group consisting of a zipper, a hook and loop fastener, and a hook.

17. The method of claim 13, wherein detaching the support surface from the support frame comprises detaching the support surface from an intermediary support coupled to the support frame, and wherein moving the support surface from the transport position to the deployed position comprises moving the intermediary support form an intermediary support transport position to an intermediary support deployed position.

18. The method of claim 13, wherein the support surface includes a set of one or more inflatable bladders, wherein the support surface is transitionable between an inflated configuration and a deflated configuration, wherein the support surface resists flexion in the inflated configuration, and wherein the support surface permits flexion in the deflated configuration; the method further comprising:
inflating the set of inflatable bladders, prior to maneuvering the patient transfer assembly towards the destination surface, to transition the support surface into the inflated configuration; and
deflating the set of inflatable bladders, after moving the support surface to the deployed position, to transition the support surface into the deflated configuration.

19. The method of claim 13, wherein moving the support surface from the transport position to the deployed position includes vertically lowering the support surface onto the destination surface.

20. The method of claim 13, wherein moving the support surface from the transport position to the deployed position includes horizontally displacing the support surface with respect to the base.
